SEQUENCE LISTING (54 sequences)

SEQ ID NO: 1 - protein, 20 aa, synthetic construct
  REGION 1..20: note = This sequence is one part of a branched amino acid sequence
KHKHHKHHKHHKHHKHHKHK

SEQ ID NO: 2 - protein, 17 aa, synthetic construct
  REGION 1..17: note = This sequence is one part of a branched amino acid sequence
KHHHKHHHKHHHKHHHK

SEQ ID NO: 3 - protein, 18 aa, synthetic construct
KHHHKHHHKHHHHKHHHK

SEQ ID NO: 4 - protein, 18 aa, synthetic construct
  SITE 1: note = D-Lysine
  SITE 5: note = D-Lysine
  SITE 9: note = D-Lysine
  SITE 14: note = D-Lysine
  SITE 18: note = D-Lysine
KHHHKHHHKHHHHKHHHK

SEQ ID NO: 5 - protein, 19 aa, synthetic construct
HKHHHKHHHKHHHHKHHHK

SEQ ID NO: 6 - protein, 20 aa, synthetic construct
HHKHHHKHHHKHHHHKHHHK

SEQ ID NO: 7 - protein, 21 aa, synthetic construct
KHHHHKHHHHKHHHHKHHHHK

SEQ ID NO: 8 - protein, 18 aa, synthetic construct
KHHHKHHHKHHHKHHHHK

SEQ ID NO: 9 - protein, 18 aa, synthetic construct
KHHHKHHHHKHHHKHHHK

SEQ ID NO: 10 - protein, 19 aa, synthetic construct
KHHHKHHHHKHHHKHHHHK

SEQ ID NO: 11 - protein, 20 aa, synthetic construct
KHKHHKHHKHHKHHKHHKHK

SEQ ID NO: 12 - protein, 17 aa, synthetic construct
KHHHKHHHKHHHKHHHK

SEQ ID NO: 13 - protein, 18 aa, synthetic construct
KHHHKHHHKHHHHKHHHK

SEQ ID NO: 14 - protein, 18 aa, synthetic construct
  SITE 1: note = D-Lysine
  SITE 5: note = D-Lysine
  SITE 9: note = D-Lysine
  SITE 14: note = D-Lysine
  SITE 18: note = D-Lysine
KHHHKHHHKHHHHKHHHK

SEQ ID NO: 15 - protein, 19 aa, synthetic construct
HKHHHKHHHKHHHHKHHHK

SEQ ID NO: 16 - protein, 20 aa, synthetic construct
HHKHHHKHHHKHHHHKHHHK

SEQ ID NO: 17 - protein, 21 aa, synthetic construct
KHHHHKHHHHKHHHHKHHHHK

SEQ ID NO: 18 - protein, 18 aa, synthetic construct
KHHHKHHHKHHHKHHHHK

SEQ ID NO: 19 - protein, 18 aa, synthetic construct
KHHHKHHHHKHHHKHHHK

SEQ ID NO: 20 - protein, 19 aa, synthetic construct
KHHHKHHHHKHHHKHHHHK

SEQ ID NO: 21 - protein, 9 aa, synthetic construct
  REGION 1..9: note = This sequence is one part of a branched amino acid sequence
HHHHNHHHH

SEQ ID NO: 22 - protein, 19 aa, synthetic construct
HHHKHHHKHHHKHHHKHHH

SEQ ID NO: 23 - protein, 4 aa, synthetic construct
HHHK

SEQ ID NO: 24 - protein, 5 aa, synthetic construct
HHKHH

SEQ ID NO: 25 - protein, 42 aa, synthetic construct
KHHHKHHHKHHHKHHHHHHKHHHKHHHKHHHKHHHHNHHHHH

SEQ ID NO: 26 - protein, 45 aa, synthetic construct
KHHHKHHHKHHHKHHHHHHKHHHKHHHKHHHKHHHHNHHHHHRGD

SEQ ID NO: 27 - protein, 33 aa, synthetic construct
HHHKHHHKHHHHHHKHHHKHHHKHHHHNHHHHH

SEQ ID NO: 28 - protein, 34 aa, synthetic construct
KHHHKHHHKHHHHHHKHHHKHHHKHHHHNHHHHH

SEQ ID NO: 29 - protein, 15 aa, synthetic construct
  REGION 1..15: note = This sequence is one part of a branched amino acid sequence
HHHKHHHKHHHKHHH

SEQ ID NO: 30 - protein, 11 aa, synthetic construct
HHHKHHHKHHH

SEQ ID NO: 31 - protein, 17 aa, synthetic construct
KHHHKHHHKHHHKHHHK

SEQ ID NO: 32 - protein, 20 aa, synthetic construct
KHKHHKHHKHHKHHKHHKHK

SEQ ID NO: 33 - protein, 19 aa, synthetic construct
  REGION 1..19: note = This sequence is one part of a branched amino acid sequence
KHKHKHKHKHKHKHKHKHK

SEQ ID NO: 34 - protein, 16 aa, synthetic construct
  REGION 1..16: note = This sequence is one part of a branched amino acid sequence
HHHKHHHKHHHKHHHK

SEQ ID NO: 35 - protein, 12 aa, synthetic construct
  REGION 1..12: note = This sequence is one part of a branched amino acid sequence
HHHKHHHKHHHK

SEQ ID NO: 36 - protein, 28 aa, synthetic construct
  SITE 16: note = Side chain of lysine linked via an amide linkage to another sequence
  SITE 26: note = Side chain of lysine linked via an amide linkage to another sequence
  SITE 27: note = Side chain of lysine linked via an amide linkage to another sequence
  SITE 28: note = Side chain of lysine linked via an amide linkage to another sequence
  SITE 28: note = Lysine carboxamide
HHHKHHHKHHHKHHHKHHHHNHHHHKKK

SEQ ID NO: 37 - protein, 25 aa, synthetic construct
  SITE 13: note = Side chain of lysine linked via an amide linkage to another sequence
  SITE 23: note = Side chain of lysine linked via an amide linkage to another sequence
  SITE 24: note = Side chain of lysine linked via an amide linkage to another sequence
  SITE 25: note = Side chain of lysine linked via an amide linkage to another sequence
  SITE 25: note = Lysine carboxamide
HHHKHHHKHHHKKHHHHNHHHHKKK

SEQ ID NO: 38 - RNA, 25 nt, synthetic construct
  source 1..25: mol_type = other RNA
gctgggatgggtttgtggagttctt

SEQ ID NO: 39 - RNA, 25 nt, synthetic construct
  source 1..25: mol_type = other RNA
gctaacaagaataaatacatgggaa

SEQ ID NO: 40 - DNA, 21 bp, synthetic construct
  source 1..21: mol_type = other DNA
  misc_feature 1..19: note = RNA
  misc_feature 20..21: note = DNA
gcaaccacgagacggcctttt

SEQ ID NO: 41 - DNA, 21 bp, synthetic construct
  source 1..21: mol_type = other DNA
  misc_feature 1..19: note = RNA
  misc_feature 20..21: note = DNA
gggatgggtttgtggagtttt

SEQ ID NO: 42 - DNA, 21 bp, synthetic construct
  source 1..21: mol_type = other DNA
  misc_feature 1..19: note = RNA
  misc_feature 20..21: note = DNA
taacaccagtacggacgggtt

SEQ ID NO: 43 - protein, 20 aa, synthetic construct
  SITE 18: note = Side chain of lysine linked via an amide linkage to another sequence
  SITE 19: note = Side chain of lysine linked via an amide linkage to another sequence
  SITE 20: note = Side chain of lysine linked via an amide linkage to another sequence
  SITE 20: note = Lysine carboxamide
KHHHKHHHKHHHKHHHKKKK

SEQ ID NO: 44 - protein, 23 aa, synthetic construct
  SITE 21: note = Side chain of lysine linked via an amide linkage to another sequence
  SITE 22: note = Side chain of lysine linked via an amide linkage to another sequence
  SITE 23: note = Side chain of lysine linked via an amide linkage to another sequence
  SITE 23: note = Lysine carboxamide
KHKHHKHHKHHKHHKHHKHKKKK

SEQ ID NO: 45 - protein, 22 aa, synthetic construct
  SITE 20: note = Side chain of lysine linked via an amide linkage to another sequence
  SITE 21: note = Side chain of lysine linked via an amide linkage to another sequence
  SITE 22: note = Side chain of lysine linked via an amide linkage to another sequence
  SITE 22: note = Lysine carboxamide
KHKHKHKHKHKHKHKHKHKKKK

SEQ ID NO: 46 - protein, 32 aa, synthetic construct
  SITE 17: note = Side chain of lysine linked via an amide linkage to another sequence
  SITE 27: note = Side chain of lysine linked via an amide linkage to another sequence
  SITE 28: note = Side chain of lysine linked via an amide linkage to another sequence
  SITE 29: note = Side chain of lysine linked via an amide linkage to another sequence
  SITE 32: note = Aspartic acid carboxamide
HHHKHHHKHHHKHHHKKHHHHNHHHHKKKRGD

SEQ ID NO: 47 - protein, 33 aa, synthetic construct
  SITE 18: note = Side chain of lysine linked via an amide linkage to another sequence
  SITE 28: note = Side chain of lysine linked via an amide linkage to another sequence
  SITE 29: note = Side chain of lysine linked via an amide linkage to another sequence
  SITE 30: note = Side chain of lysine linked via an amide linkage to another sequence
  SITE 33: note = Aspartic acid carboxamide
KHHHKHHHKHHHKHHHKKHHHHNHHHHKKKRGD

SEQ ID NO: 48 - protein, 24 aa, synthetic construct
  SITE 17: note = Side chain of lysine linked via an amide linkage to another sequence
  SITE 19: note = Side chain of lysine linked via an amide linkage to another sequence
  SITE 20: note = Side chain of lysine linked via an amide linkage to another sequence
  SITE 21: note = Side chain of lysine linked via an amide linkage to another sequence
  SITE 24: note = Aspartic acid carboxamide
HHHKHHHKHHHKHHHKKGKKKRGD

SEQ ID NO: 49 - protein, 25 aa, synthetic construct
  SITE 16: note = Side chain of lysine linked via an amide linkage to another sequence
  REGION 1..25: note = This sequence is one part of a branched amino acid sequence
HHHKHHHKHHHKHHHKHHHHNHHHH

SEQ ID NO: 50 - protein, 26 aa, synthetic construct
  SITE 17: note = Side chain of lysine linked via an amide linkage to another sequence
  REGION 1..26: note = This sequence is one part of a branched amino acid sequence
HHHKHHHKHHHKHHHKKHHHHNHHHH

SEQ ID NO: 51 - protein, 27 aa, synthetic construct
  SITE 18: note = Side chain of lysine linked via an amide linkage to another sequence
  REGION 1..27: note = This sequence is one part of a branched amino acid sequence
KHHHKHHHKHHHKHHHKKHHHHNHHHH

SEQ ID NO: 52 - protein, 18 aa, synthetic construct
  SITE 17: note = Side chain of lysine linked via an amide linkage to another sequence
  REGION 1..18: note = This sequence is one part of a branched amino acid sequence
HHHKHHHKHHHKHHHKKG

SEQ ID NO: 53 - protein, 22 aa, synthetic construct
  SITE 13: note = Side chain of lysine linked via an amide linkage to another sequence
  REGION 1..22: note = This sequence is one part of a branched amino acid sequence
HHHKHHHKHHHKKHHHHNHHHH

SEQ ID NO: 54 - protein, 28 aa, synthetic construct
  SITE 13: note = Side chain of lysine linked via an amide linkage to another sequence
  SITE 23: note = Side chain of lysine linked via an amide linkage to another sequence
  SITE 24: note = Side chain of lysine linked via an amide linkage to another sequence
  SITE 25: note = Side chain of lysine linked via an amide linkage to another sequence
  SITE 28: note = Aspartic acid carboxamide
HHHKHHHKHHHKKHHHHNHHHHKKKRGD

(19) United States
(12) Patent Application Publication
(10) Pub. No.: US 2023/0121879 A1
(43) Pub. Date: Apr. 20, 2023

(54) METHODS FOR PREPARING NANOPARTICLE COMPOSITIONS CONTAINING HISTIDINE-LYSINE COPOLYMERS

(71) Applicant: Sirnaomics, Inc., Gaithersburg, MD (US)

(72) Inventors: Wookhyun KIM, Gaithersburg, MD (US); Daniel MUTISYA, Gaithersburg, MD (US); David M. EVANS, Gaithersburg, MD (US)

(21) Appl. No.: 17/951,842

(22) Filed: Sep. 23, 2022

Related U.S. Application Data
(63) Continuation of application No. PCT/US2022/076867, filed on Sep. 22, 2022.
(60) Provisional application No. 63/247,290, filed on Sep. 22, 2021.

(51) Int. Cl. A61K 31/713; A61K 9/51

(52) U.S. Cl. CPC A61K 31/713; A61K 9/5146; A61K 9/5192; B82Y 5/00

(57) ABSTRACT

Improved pharmaceutical nanoparticle compositions and improved methods for preparing the compositions comprising histidine-lysine copolymers and an acetate salt or phosphate anion are provided. The addition of acetate or phosphate anion to the histidine-lysine copolymer prior to mixing with a nucleic acid alters nanoparticle size and polydispersity index of the compositions and provides a more uniform particle size distribution.

### CROSS-REFERENCE TO RELATED PATENT APPLICATION

This application is a continuation of international application PCT/US2022/76867, filed Sep. 22, 2022, which claims the benefit of and priority to U.S. Provisional Patent Application No. 63/247,290, filed Sep. 22, 2021, which application is incorporated herein by reference in its entirety.

### SEQUENCE LISTING

The instant application contains a Sequence Listing which has been submitted electronically in ST.26 (XML) format and is hereby incorporated by reference in its entirety. Said Sequence List, created on Dec. 8, 2022, is named 4690_0051 i_SL_ST_26 and is 77 kilobytes in size.

### FIELD

Improved pharmaceutical nanoparticle compositions comprising histidine-lysine copolymers and either acetate or phosphate anion are provided. Improved methods for preparing the nanoparticle compositions also are provided. Addition of acetate or phosphate anion alters the nanoparticle size and polydispersity index of the compositions and provides a more uniform particle size distribution.

### SUMMARY

The disclosed embodiments provide a histidine-lysine copolymer to which acetate or phosphate anion is added, modifying the properties of the nanoparticles.

Pharmaceutical compositions containing siRNA molecules are provided comprising a histidine-lysine copolymer. The particles are formed by microfluidic mixing of a solution comprising the nucleic acid and a solution comprising the copolymer, where acetate and, optionally, phosphate, is added to the copolymer solution. Upon mixing the solutions spontaneously form nanoparticles containing the nucleic acid and the copolymer. The presence of the acetate or phosphate anion is to reduce the size of the nanoparticles and produces a size distribution of the resulting nanoparticles more uniform with a lower Polydispersity Index (PDI).

In some embodiments, the acetate content ranges between about 11 and about 20 percent (w/w) of the copolymer solution used to prepare the composition. In other embodiments, the phosphate anion content ranges between about 1 and about 2 mM of the composition. Nanoparticle size and PDI is reduced, especially with acetate or phosphate anion content at the lower end of the range, permitting more efficient transfection of the siRNA into cells of the recipient subject. In certain embodiments, nanoparticle diameter range is between about 100 and about 150 nm, while the PDI ranges in some embodiments between about 0.03 and about 0.28.

What is provided is a pharmaceutical composition containing a nanoparticle formulation of a histidine-lysine copolymer and an effective amount of a nucleic acid, formed by microfluidic mixing of a solution containing the nucleic and a solution containing the copolymer. The copolymer solution used to prepare the nanoparticle formulation contains an acetate salt present in the amount of between about 11% and about 20%. In the nanoparticle formulation about at least 40%, at least 45%, at least 50%, at least 55% or at least about 60% of the nanoparticles formed have a diameter in a range selected from the group consisting of between about 40 and about 200 nm, between about 50 and about 150 nm, between about 50 and about 100 nm and between about 60 and about 90 nm. The nanoparticles in the composition have a polydispersity index (PDI) selected from the group consisting of between about 0.4 and about 0.3, between about 0.3 and about 0.2, between about 0.2 and about 0.1, between about 0.1 and about 0.05, between about 0.05 and about 0.03, or between about 0.03 and about 0.01. The histidine-lysine copolymer may, for example, be selected from the group consisting of HKP, HKP(+H), HKP, HKP(+H), H³K4b, and H³K8b. The nucleic acid may be an siRNA molecule and may be, for example, 18-25 nucleotides long. In a specific embodiment, the siRNA reduces the expression of TGFβ1. The histidine-lysine copolymer may comprise, for example, HKP(+H) or HKP.

Also provided are methods of preparing a pharmaceutical composition as described above, by mixing a solution (a) comprising a nucleic acid, and a solution (b) comprising a histidine-lysine copolymer and acetate or phosphate anion, where the nucleic acid solution (a) comprises at least one siRNA, and where the histidine-lysine copolymer solution (b) has an acetate content of 11 to about 20 percent (or phosphate anion content of about 1-2 mM). In certain embodiments, the ratio of the copolymer to the nucleic acid is about 2.5 to about 1 (w/w). The solution (b) has, for example, an acetate content selected from the group consisting of between about 11 and about 20 percent; between about 17 and about 20 percent; between about 14 and about 17 percent; between about 12 and about 14 percent; and between about 11 and about 14 percent.

Further provided are methods of treating a subject having a disease by administering to the subject an effective amount of a pharmaceutical composition as described above, where the nucleic acid molecule is an RNA molecule that modulates the production of a protein or peptide of interest, and where the infection is ameliorated by the administration of the pharmaceutical composition. The RNA molecule advantageously contains one or more siRNA molecules that inhibit expression of one or more genes associated with the disease. The disease may be cancer, for example, isSCC, BCC, H&N, liver, NSCLC, other solid tumors, pancreatic, colon, breast, prostate or CNS tumors. The disease may be an infection. The subject may be a mammal, such as a human.

### BRIEF DESCRIPTION OF THE DRAWINGS

FIG. 1 (a)-1(g) are examples of histidine-lysine copolymer structures that may be used in the disclosed embodiments. (a): H³K4b; (b): H²K4b; (c): HK4b; (d): H³K8b_{(+RGD)}; (e): H³K8b_{(+RGD)} or _{(K+)}H³K8b_{(+RGD)}; (f): H³K(G)8b; and (g): _{(-HHHK)}H³K8b or _{(-HHHK)}H³K8b_{(+RGD)}

FIG. 2 shows results of forming nanoparticles in the presence of HKP and different concentrations of acetate.

FIG. 3 shows results of forming nanoparticles in the presence of HKP(+H) and different concentrations of acetate

FIG. 4 shows the effect of storage at 4° C. and −20° C. on nanoparticle size and PDI after addition of phosphate anion at three HKP(+H):siRNA ratios: 1.5:1, 2.0:1 and 2.5:1

FIGS. 5 (a) through 5 (d) show the effect of phosphate anion on HKP(+H) nanoparticle size and Polydispersity Index (PDI): (a): table showing values for samples evaluated at various Na₂PO₄ concentrations (b): graphs showing samples evaluated with HKP ratio of 2.5:1 using 2 nM Na₂PO₄; (c): graphs showing samples evaluated with HKP ratio of 2.0:1 using 2 nM Na₂PO (d): graphs showing samples evaluated with HKP ratio of 1.5:1 using 2 nM Na₂PO.

FIG. 6 shows the effect of phosphate anion on Zeta potential.

### DETAILED DESCRIPTION

Pharmaceutical compositions are described containing nanoparticles formed by mixing a histidine-lysine copolymer and a siRNA molecule in the presence of acetate. Methods of forming the compositions are provided, together with methods of using the compositions to treat disease by inhibiting gene expression in recipient subjects.

More specifically, it has been found that addition of acetate or phosphate anion to the copolymer solution prior to nanoparticle formation alters nanoparticle properties such as nanoparticle diameter and Polydispersity index, providing particles of more uniform and more desirable size, resulting in greater or more efficient delivery of the siRNA molecule to the target cell(s). Formation of nanoparticle compositions in the presence of an acetate, advantageously ammonium acetate, an in amount between of about 11 to about 20 percent of the composition (or phosphate anion added in the amount of 1-2 mM) results in a composition where the nanoparticles have a more favorable size distribution. For example, at least 40%, at least 45%, at least 50%, at least 55% or at least about 60% of the nanoparticles formed in the presence of acetate have a diameter in a range selected from the group consisting of between about 40 and about 200 nm, between about 50 and about 150 nm, between about 50 and about 100 nm, and between about 60 and about 90 nm.

### Histidine-Lysine (HK) Polypeptides

Effective means for transferring nucleic acids into target cells are important tools, both in the basic research setting and in clinical applications. A diverse array of nucleic acid carriers is currently required because the effectiveness of a particular carrier depends on the characteristics of the nucleic acid that is being transfected [Blakney et al. Biomacromolecules 2018, 19: 2870-2879. Goncalves et al. Mol Pharm 2016; 13: 3153-3163. Kauffman et al. Biomacromolecules 2018; 19: 3861-3873. Peng et al. Biomacromolecules 2019; 20: 3613-3626. Scholz et al. J Control Release 2012; 161: 554-5651. Among various carriers, non-viral delivery systems have been developed and reported to be more advantageous than the viral delivery system in many aspects [Brito et al. Adv Genet. 2015; 89: 179-233]. For example, the large molecular weight branched polyethylenimine (PEI, 25 kDa) is an excellent carrier for plasmid DNA but not for mRNA. However, by decreasing the molecular weight of PEI to 2 kDa, it becomes a more effective carrier of mRNA [Bettinger et al. Nucleic Acids Res 2001; 29: 3882-38911

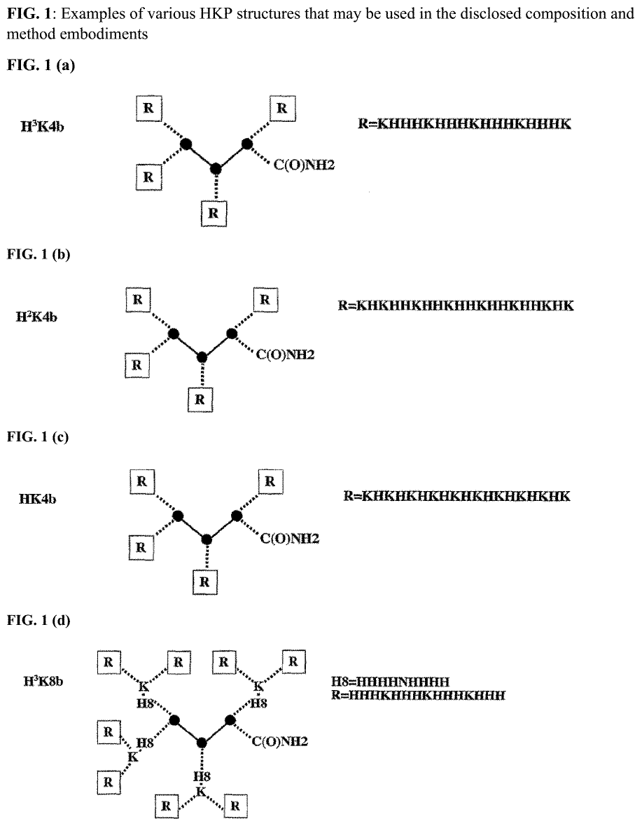

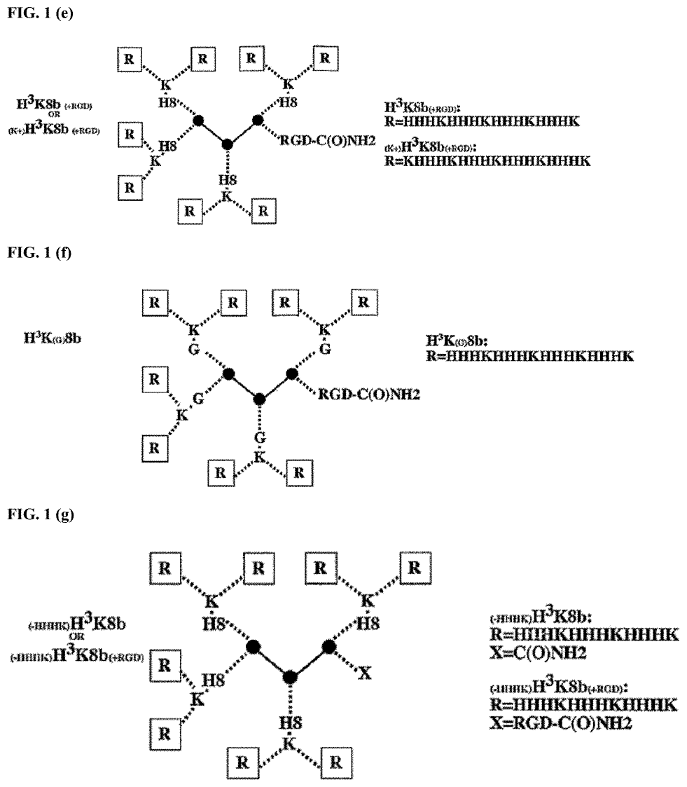

The four-branched histidine-lysine (HK) peptide polymer H²K4b has been shown to be a good carrier of large molecular weight DNA plasmids [Leng et al. Nucleic Acids Res 2005; 33: e40.1, but a poor carrier of relatively low molecular weight siRNA [Leng et al. J Gene Med 2005; 7: 977-986.]. Two histidine-rich peptides analogs of H²K4b, namely H³K4b and H³K(+H)4b, were shown to be effective carriers of siRNA [Leng et al. J Gene Med 2005; 7: 977-986. Chou et al. Biomaterials 2014; 35: 846-8551, although H³K(+H)4b appeared to be modestly more effective [Leng et al. Mol Ther 2012; 20: 2282-22901 Moreover, the H³K(+H)4b carrier of siRNA induced cytokines to a significantly lesser degree in vitro and in vivo than H³K4b siRNA polyplexes [Leng et al. Mol Ther 2012; 20: 2282-2290], which were already at very low levels. Suitable HK polypeptides are described in WO/2001/047496, WO/2003/090719, and WO/2006/060182, the contents of each of which are incorporated herein in their entireties. These polypeptides have a lysine backbone (three lysine residues) where the lysine side chain ε-amino groups and the N-terminus are coupled to various HK sequences. HK polypeptide carriers can be synthesized by methods that are well-known in the art including, for example, solid-phase peptide synthesis (SPPS). FIG. 1 shows several HK polymer structures that can be used in the disclosed composition and method embodiments.

It was found that such histidine-lysine peptide polymers (“HK polymers”), in addition to their ability to package and carry siRNAs also were surprisingly effective as mRNA carriers, and that they can be used, alone or in combination with liposomes, to provide effective delivery of mRNA into target cells. Similar to PEI and other carriers, initial results suggested HK polymers differ in their ability to carry and release nucleic acids. However, because HK polymers can be reproducibly made on a peptide synthesizer, their amino acid sequence can be easily varied, thereby allowing fine control of the binding and release of siRNAs, miRNAs or mRNAs, as well as the stability of polyplexes containing the HK polymers and mRNA [Chou et al. Biomaterials 2014; 35: 846-855. Midoux et al. Bioconjug Chem 1999; 10: 406-411. Henig et al. Journal of American Chemical Society 1999; 121: 5123-51261 When siRNA, miRNA, or mRNA molecules are admixed with one or more HKP carriers the components self-assemble into nanoparticles.

As described herein, advantageously the HK polymer comprises four short peptide branches linked to a three-lysine amino acid core. The peptide branches consist of histidine and lysine amino acids, in different configurations. The general structure of these histidine-lysine peptide polymers (HK polymers) is shown in Formula I, where R represents the peptide branches and K is the amino acid L-lysine.

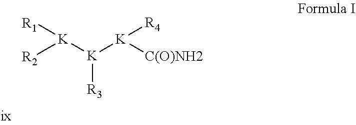

In Formula I where K is L-lysine and each of R₁, R₂, R₃ and R₄ is independently a histidine-lysine peptide. The R₁₋₄ branches may be the same or different in the HK polymers of the invention. When a R branch is “different”, the amino acid sequence of that branch differs from each of the other R branches in the polymer. Suitable R branches used in the HK polymers of the invention shown in Formula I include, but are not limited to, the following R branches R_{A}-R_{-J}:

(SEQ ID NO: 1)
R_{A} = KHKHHKHHKHHKHHKHHKHK-
(SEQ ID NO: 2)
R_{B} = KHHHKHHHKHHHKHHHK-
(SEQ ID NO: 3)
R_{C} = KHHHKHHHKHHHHKHHHK-
(SEQ ID NO: 4)
R_{D} = kHHHkHHHkHHHHkHHHk-
(SEQ ID NO: 5)
R_{E} = HKHHHKHHHKHHHHKHHHK-
(SEQ ID NO: 6)
R_{F} = HHKHHHKHHHKHHHHKHHHK-
(SEQ ID NO: 7)
R_{G} = KHHHHKHHHHKHHHHKHHHHK-
(SEQ ID NO: 8)
R_{H} = KHHHKHHHKHHHKHHHHK-
(SEQ ID NO: 9)
R_{I} = KHHHKHHHHKHHHKHHHK-
(SEQ ID NO: 10)
R_{J} = KHHHKHHHHKHHHKHHHHK-

Specific HK polymers that may be used in the siRNA, miRNA and/or mRNA compositions include, but are not limited to, HK polymers where each of R1, R2, R3 and R4 is the same and selected from R_{A}-R_{J} (Table 1). These HK polymers are termed H²K4b, H³K4b, H³K(+H)4b, H³k(+H)4b, H-H³K(+H)4b, HH-H³K(+H)4b, H⁴K4b, H³K(1+H)4b, H³K(3+H)4b and H³K(1,3+H)4b, respectively. In each of these 10 examples, upper case “K” represents a L-lysine, and lower case “k” represents D-lysine. Extra histidine residues, in comparison to H³K4b, are underlined within the branch sequences. Nomenclature of the HK polymers is as follows:

1) for H³K4b, the dominant repeating sequence in the branches is -HHHK-, thus “H³K” is part of the name; the “4b” refers to the number of branches;
2) there are four -HHHK- motifs in each branch of H³K4b and analogues; the first -HHHK-motif (“1”) is closest to the lysine core;
3) H³K(+H)4b is an analogue of H³K4b in which one extra histidine is inserted in the second -HHHK- motif (motif 2) of H³K4b;
4) for H³K(1+H)4b and H³K(3+H)4b peptides, there is an extra histidine in the first (motif 1) and third (motif 3) motifs, respectively;
5) for H³K(1,3+H)4b, there are two extra histidine residues in both the first and the third motifs of the branches.

TABLE 1
Examples of branched polymers

| | | Sequence |
|---|---|---|
| Polymer | Branch Sequence | Identifier |
| H²K4b | R_{A} = KHKHHKHHKHHKHHKHHKHK- | 11 |
| H³K4b | 4 3 2 1 | 12 |
| | R_{B} = KHHHKHHHKHHHKHHHK- | |
| H³K(+H)4b | R_{C} = KHHHKHHHKHHHHKHHHK- | 13 |
| H³k(+H)4b | R_{D} = kHHHkHHHkHHHHkHHHk- | 14 |
| H-H³K(+H)4b | R_{E} = HKHHHKHHHKHHHHKHHHK- | 15 |
| HH-H³K(+H)4b | R_{F} = HHKHHHKHHHKHHHHKHHHK- | 16 |
| H⁴K4b | R_{G} = KHHHHKHHHHKHHHHKHHHHK- | 17 |
| H³K(1+H)4b | R_{H} = KHHHKHHHKHHHKHHHHK- | 18 |
| H³K(3+H)4b | R_{I} = KHHHKHHHHKHHHKHHHK- | 19 |
| H³K(1,3+H)4b | R_{J} = KHHHKHHHHKHHHKHHHHK- | 20 |

TABLE 2
Additional examples of HK Polymers (duplication?)

| | SEQ |
|---|---|
| | ID |
| Peptide Sequence | No. |
| HHHHNHHHH | 21 |
| HHHKHHHKHHHKHHHKHHH | 22 |
| HHHK | 23 |
| HHKHH | 24 |
| KHHHKHHHKHHHKHHHHHHKHHHKHHHKHHHKHHHHNHHHHH | 25 |
| KHHHKHHHKHHHKHHHHHHKHHHKHHHKHHHKHHHHNHHHHHRGD | 26 |
| HHHKHHHKHHHHHHKHHHKHHHKHHHHNHHHHH | 27 |
| KHHHKHHHKHHHHHHKHHHKHHHKHHHHNHHHHH | 28 |
| HHHKHHHKHHHKHHH | 29 |
| HHHKHHHKHHH | 30 |
| KHHHKHHHKHHHKHHHK | 31 |
| KHKHHKHHKHHKHHKHHKHK | 32 |
| KHKHKHKHKHKHKHKHKHK | 33 |
| HHHKHHHKHHHKHHHK | 34 |
| HHHKHHHKHHHK | 35 |
| H³K8b | 36 |
| (-HHHK) H³K8b | 37 |

Methods well known in the art, including gel retardation assays, heparin displacement assays and flow cytometry can be performed to assess performance of different formulations containing HK polymer plus liposome in successfully delivering mRNA. Suitable methods are described in, for example, Gujrati et al., Mol. Pharmaceutics 11:2734-2744 (2014), Parnaste et al., Mol Ther Nucleic Acids. 7: 1-10 (2017).

Detection of nucleic acid uptake into cells can also be achieved using SmartFlare® technology (Millipore Sigma). These smart flares are beads that have a sequence attached that, when recognizing the RNA sequence in the cell, produce an increase in fluorescence that can be analyzed with a fluorescent microscope. siRNAs can reduce expression of a target gene while mRNA can increase it. miRNAs can either increase or decrease expression.

Other methods include measuring protein expressions from the nucleic acid, for example, an mRNA encoding luciferase can be used to measure the efficiency of transfection using methods that are well known in the art. See, for example, this was accomplished with luciferase mRNA in a recent publication (He et al, J Gene Med. 2021 February; 23(2):e3295) to demonstrate the efficacy of delivering mRNA using a HKP and liposome formulation.

### Additions of Acetate or Phosphate Anion

In disclosed siRNA/HKP composition embodiments, ammonium acetate added to the HKP composition in the pharmaceutical compositions in a range between about 11 and about 20 percent (w/w). Ammonium acetate, unlike other, similar compounds is lyophilizable; upon dry down, acetate permits the (pharmaceutical composition) product to remain intact. Example 1 describes an experiment where acetate content was varied between 11 and 25 percent of the composition. As the acetate content was lowered, the nanoparticles were smaller and more uniform, and the nanoparticles had a lower PDI. See FIGS. 1 through 3.

In some embodiments the histidine-lysine solution may comprise acetate where the acetate is present in an amount selected from the group consisting of: between about 11 and about 20 percent; between about 17 and about 20 percent; between about 14 and about 17 percent; between about 12 and about 14 percent; and between about 11 and about 14 percent.

Phosphate anion also may be added (at about 1 to about 2 mM) to the histidine-lysine copolymer of the disclosed embodiments to affect nanoparticle diameter and PDI, with the same benefit as seen with acetate.

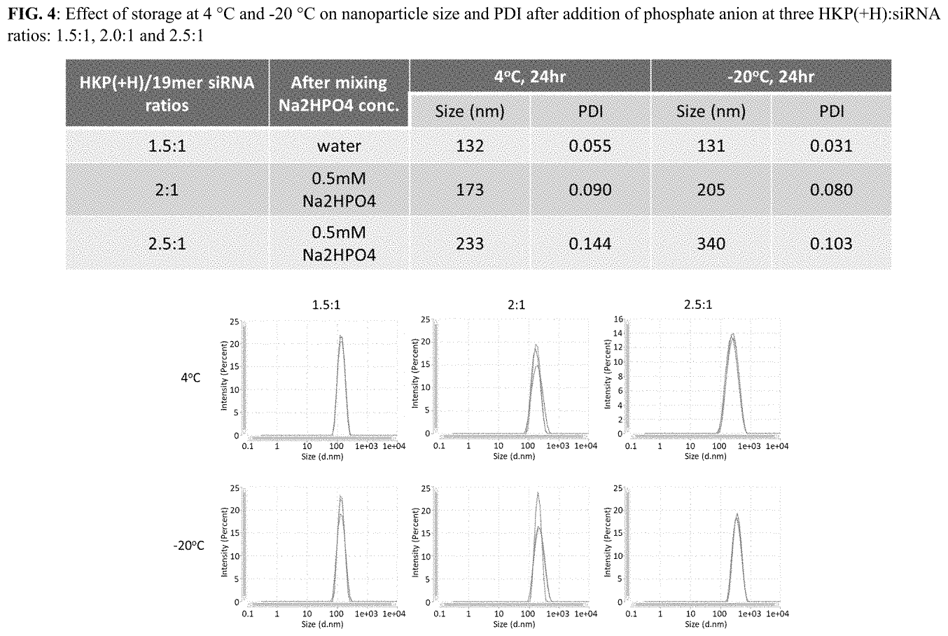

In some embodiments phosphate anion is added to the pharmaceutical composition (see, e.g., Example 2). Example 2 and the accompanying figures show the effect of adding phosphate anion to separate composition samples, resulting in reduced nanoparticle diameters and PDI. The addition of about 1 to about 2 mM phosphate anion to the composition comprising HKP(+H) reduced nanoparticle size to below 150 nm, while PDI remain between about 0.04 and 0.08. When these solutions were stored at 4° C. or −20° C., PDI remained below 0.1 in 4 degrees, while PDI dropped further to 0.03 at −20 degrees for 24 hours and the lowest ratio of polymer to siRNA (1.5:1). At higher ratios, PDI increased. See FIGS. 4-6.

Nucleic Acids—siRNA, miRNA and mRNA

The nucleic acids used in the disclosed embodiments comprise siRNA, miRNA and mRNA molecules that target genes of interest in a variety of conditions and diseases are well known in the art. Certain disclosed embodiments include at least one nucleic acid—e.g., siRNA—in each composition, for example, as disclosed in published U.S. Pat. No. 9,642,873. In some embodiments, the gene-targeting siRNA comprises a sense strand and an antisense strand, each containing a core sequence that is 19, 21, 23 or 25 nucleotides in length. The sense and antisense strands of siRNA typically anneal to form a duplex. Within the complementary duplex region, the sense strand core sequence is 100% complementary to the antisense core sequence. In some embodiments the siRNAs can be asymmetric where one strand is shorter than the other (typically by 2 bases e.g. a 21 mer with a 23 mer or a 19 mer with a 21 mer or a 23 mer with a 25 mer). The strands may be modified by inclusion of a dTdT overhang group on the 3′ end of selected strands.

In the disclosed embodiments, siRNA, miRNA and mRNA molecules may be designed and selected to target the sequences of any number of genes of interest, e.g., various strains of a virus as well as their mutants.

In some embodiments, double stranded siRNA may be unmodified or chemically modified at the 2′ position with 2′—OCH₃, (or 2′-OMe) or by 2′-F, and/or at the 5′ position with —P(O)₂═S, —P(S)₂═O. Other chemical modifications, such as pegylation or lipid functionalization may be used to improve the overall stability and bioavailability of the RNAi.

In some embodiments, siRNA duplexes are capable of targeting multiple genes with a single effector sequence.

In some embodiments in each of the siRNA, miRNA, and mRNA molecules, one or more of the nucleotides in either the sense or the antisense strand can be a modified nucleotide. Modified nucleotides can improve stability and decrease immune stimulation by the siRNAs. The modified nucleotide may be, for example, a 2′-O-methyl, 2′-methoxyethoxy, 2′-fluoro, 2′-allyl, 2′-O[2-(methylamino)-2-oxoethyl], 4′-thio, 4′-CH2-O-2′-bridge, 4′-(CH2)2-O-2′-bridge, 2′-LNA, 2′-amino or 2′-O-(N-methylcarbamate) ribonucleotide. In other embodiments, one or more of the phosphodiester linkages between the ribonucleotides may be modified to improve resistance to nuclease digestion. Suitable modifications include the use of phosphorothioate and/or phosphorodithioate modified linkages.

Nucleic acids that can be used in the pharmaceutical compositions of various embodiments include the following nonlimiting examples. Many other miRNA, mRNA and siRNA molecules may be used in the disclosed embodiments. Such molecules are known in the art.

RNA Sequence Sense Strand

(SEQ ID NO: 38)
hmMCL1_1 5′-GCUGGGAUGGGUUUGUGGAGUUCUU-3′
(SEQ ID NO: 39)
hmMCL1_2 5′-GCUAACAAGAAUAAAUACAUGGGAA-3′
(SEQ ID NO: 40)
hmMCL1_3 5′-GCAACCACGAGACGGCCUU-dTdT-3′
(SEQ ID NO: 41)
hmMCL1_4 5′-GGGAUGGGUUUGUGGAGUU-dTdT-3′
(SEQ ID NO: 42)
hmMCL1_5 5′-UAACACCAGUACGGACGGG-dTdT-3′

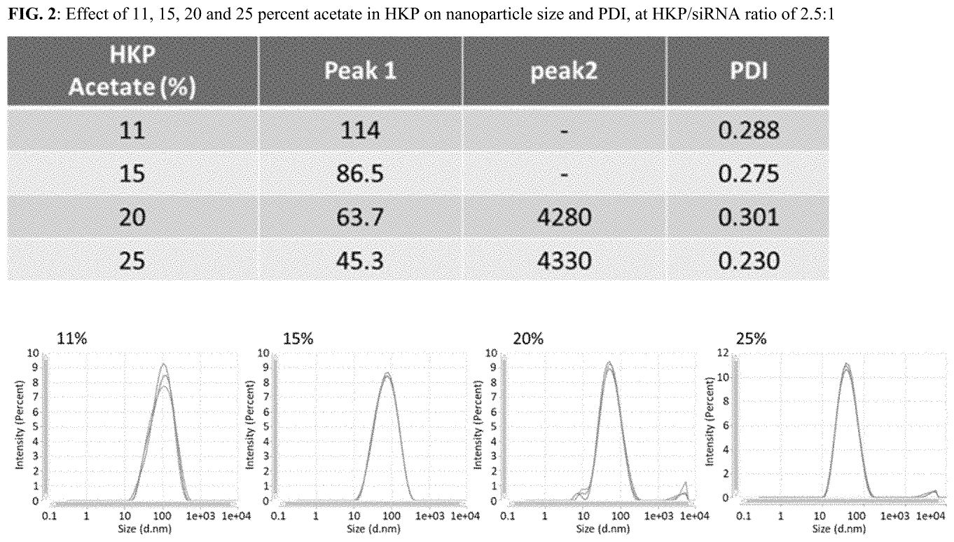

Sequence hmMCL1_5 has previously been described (Zhang et al., J. Biol. Chem., 277:37430-37438 (2002)). As shown in FIG. 2, sequences hmMCL1_1, hmMCL1_2, hmMCL1_3 and hmMCL1_4 showed excellent activity in silencing the MCL1 gene in FaDu cells, which is a cell line derived from a squamous cell carcinoma of the hypopharynx.

### Determination of Efficacy of the Nucleic Acids

Depending on the particular target RNA sequences and the dose of the nanoparticle composition delivered, partial or complete loss of function for the target RNAs may be observed. A reduction or loss of RNA levels or expression (either RNA expression or encoded polypeptide expression) in at least 50%, 60%, 70%, 80%, 90%, 95% or 99% or more of targeted cells is exemplary. Inhibition of target RNA levels or expression refers to the absence (or observable decrease) in the level of RNA or RNA-encoded protein. Specificity refers to the ability to inhibit the target RNA without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). Inhibition of target RNA sequence(s) by the dsRNA agents of the invention also can be measured based upon the effect of administration of such dsRNA agents upon development/progression of a target RNA-associated disease or disorder, e.g., tumor formation, growth, metastasis, etc., either in vivo or in vitro. Treatment and/or reductions in tumor or cancer cell levels can include halting or reduction of growth of tumor or cancer cell levels or reductions of, e.g., 10%, 20%, 30%, 40%, 50%, 60%, 70%, 80%, 90%, 95% or 99% or more, and can also be measured in logarithmic terms, e.g., 10-fold, 100-fold, 1000-fold, 105-fold, 106-fold, or 107-fold reduction in cancer cell levels could be achieved via administration of the nanoparticle composition to cells, a tissue, or a subject. The subject may be a mammal, such as a human.

### Definitions

As used herein, “a” or “an” may mean one or more. As used herein, “another” may mean at least a second or more.

The term “amino acid” is inclusive of the 20 common amino acids, as well as “nonstandard amino acids,” for example, D-amino acids and chemically (or biologically) produced derivatives of “common” amino acids, including for example, beta-amino acids.

A compound is “associated with” a second compound if the two compounds have formed a complex as a result of covalent or non-covalent interactions between the two compounds.

The term “copolymer” refers to a polymer that contains two or more types of units, regardless of the arrangement of units along the chain (random, alternating, block, graft), and regardless of its molecular structure (linear or branched). The term “histidine copolymer” means that the copolymer comprises histidine as one of its unit types. The term “transport polymer” means a polymer comprising the histidine copolymer of the disclosed embodiments.

The term “branch” is inclusive of any monomer or linear polymer (including co-polymer) thereof, which is covalently attached at least one end to the side group of a branching monomer. A branch which itself comprises one or more branching monomers is referred to as a “non-terminal branch”. A branch which does not comprise a branching monomer is referred to as a “terminal branch”. A “terminal branch” may include for example, the final division of branching of histidine or lysine to the n-terminal amino acid of the branch. The terminal branch may include a non-histidine or lysine amino acid (e.g., a cysteine or other linking agent), which aids in conjugating a stabilizing agent (such as PEG or HPMA) and/or a targeting ligand.

The term “branched polymer” is inclusive of any polymer comprising at least one backbone and at least one terminal branch. A branched polymer may further comprise one or more non-terminal branches.

The terms “HK peptide,” “HK polymer,” and “HK carrier” are intended to mean transport polymers, which include histidine and lysine, including the polymers encompassed by the disclosed embodiments.

The term “in vivo” includes therapy based on injection, whether intravenous or local (e.g., intratumoral, intramuscular, subcutaneous, intratracheal, intravenous, or intraocular injection into organ or airway directly, injection into vessels of the organ, or aerosolized into airways). The term “in vivo” also includes therapy based on electroporation of tumor, tissue, or organ.

The term “lipid” is used as it is in the art and includes any chemical species having a hydrophobic and a hydrophilic portion. Hydrophilic characteristics typically derive from the presence of phosphato, carboxylic, sulfato, amino, sulfhydryl, nitro, and other like groups. Hydrophobicity may be conferred by cholesterol and derivatives thereof and by the inclusion of groups that include, but are not limited to, long chain saturated and unsaturated aliphatic hydrocarbon groups and such groups substituted by one or more aromatic, cycloaliphatic or heterocyclic group(s).

The term “non-cationic lipid” refers to any of a number of lipid species that exist either in an uncharged form a neutral zwitterionic form, or an anionic form at physiological pH. Such lipids include, for example diacylphosphatidylcholine, diacylphosphatidylethanolamine, ceramide, sphingomyelin, cephalin, cardiolipin, cerebrosides, DOPE, and cholesterol.

The term “cationic lipid” refers to any of a number of lipid species which carries a net positive charge at physiologic pH. Such lipids include, but are not limited to, DODAC, DOTMA, DDAB, DOSPER, DOSPA, DOTAP, DC-Chol and DMRIE. Additionally, a number of commercial preparations of cationic lipids are available which can be used in the disclosed embodiments. These include, for example, LIPOFECTIN® (commercially available cationic liposomes comprising DOTMA and DOPE, from GIBCO/BRL, Grand Island, N.Y., USA); LIPOFECTAMINE® (commercially available cationic liposomes comprising DOSPA and DOPE, from GIBCO/BRL); and TRANSFECTAM® (commercially available cationic liposomes comprising DOGS from Promega Corp., Madison, Wis., USA).

The term “Polydispersity Index,” or PDI, refers to the heterogeneity of a sample of nanoparticles. PDI is independent of nanoparticle size; rather, the lower the PDI, the more homogeneous the size of nanoparticles in the sample, regardless of size. (ISO standards ISO 22,412, Particle size analysis—Dynamic light scattering (DLS))

The term “peptide” is inclusive of both straight and branched amino acid chains, as well as cyclic amino acid chains, which comprise at least 2 amino acid residues. The terms “peptide” and “polypeptide” are used interchangeably herein.

A “pharmaceutical agent” includes any therapeutic agent useful in preventing, delaying or reducing the severity of the onset of a disease, or in reducing the severity of an ongoing disease, or in enhancing normal physiological functioning, as well as diagnostic agents, for example, a marker gene (GFP, luciferase). A “pharmaceutical agent” may consist of one or more therapeutic agents, one or more diagnostic agents, or a combination of one or more therapeutic and one or more diagnostic agents.

As used herein, a “pharmaceutically acceptable” component (such as a salt, carrier, excipient or diluent) of a pharmaceutical agent delivery composition according to the present disclosed embodiments is a component which (1) is compatible with the other ingredients of the delivery composition in that it can be included in the delivery composition without eliminating the capacity of the composition to deliver the pharmaceutical agent; and (2) where the delivery composition is intended for therapeutic uses, is suitable for use with an animal (e.g., a human) without undue adverse side effects, such as toxicity, irritation, and allergic response. Side effects are “undue” when their risk outweighs the benefit provided by the pharmaceutical agent.

As used herein, the term “physiologic pH” is defined as a pH between about 7.2 and about 7.5.

As used herein, the term “recombinant” means a cell having genetically engineered DNA, which was prepared in vitro and includes DNA from the host organism or, more often, from a different species, genus, family, order or class as compared to the host organism.

The term “siRNA” is used as it is in the art, and includes a duplex of RNA (19 to 25) bases or fewer in each strand) that targets mRNA. siRNA may be chemically or enzymatically synthesized. siRNA in accordance with the present disclosed embodiments may be incorporated and then activated in RISC (RNA-induced silencing complex).

A “therapeutically effective amount” is an amount necessary to prevent, delay or reduce the severity of the onset of disease, or an amount necessary to arrest or reduce the severity of an ongoing disease, and also includes an amount necessary to enhance normal physiological functioning.

The word “transfect” is broadly used herein to refer to introduction of an exogenous compound, such as a polynucleotide sequence, into a prokaryotic or eukaryotic cell; the term includes, without limitation, introduction of an exogenous nucleic acid into a cell, which may result in a permanent or temporary alteration of genotype in an immortal or non-immortal cell line.

A number of patterns of HK polymers that might be effective for siRNA, miRNA or mRNA transport were isolated, developed and evaluated. Among the polymers with 4 branches, the repeating pattern of HHHK (e.g., H³K4b on the terminal branch appears to augment uptake of siRNA more effectively than the repeating patterns of HHK (e.g., H²K4b) or HK (e.g., HK4b). As a result, a similar pattern was adopted in constructing the highly branched H³K8b and found it to be highly effective for preparing carriers of siRNA.

H³K8b has eight terminal branches, and has a high percentage of histidine residues and a low percentage of lysine residues. Compared to HHK, the pattern HHHK has an increased buffering capacity because of the higher ratio of histidine residues, and reduced binding because of the lower ratio of lysine residues. An increased number of histidine residues in the terminal branches that buffer the acidic endosomal compartment would allow endosomal lysis and escape of DNA from the endosomes. Similarly, the histidine rich domain in H³K8b would be expected to increase cytosol delivery by enhancing the buffering capacity of the polymer. Nevertheless, replacement of the histidine-rich domain with a glycine or a truncated histidine-rich domain (-HHKHH) resulted in HK polymers that were ineffective carriers of siRNA. That the HK polymer with the truncated histidine rich domain was no more effective than the polymer with the glycine suggest that the buffering capacity of the histidine-rich domain may not be a dominant mechanism for this domain. Moreover, these results indicate that all the domains (the terminal branches and the histidine-rich domain) of the highly branched HK peptides are important for the development of an effective siRNA carrier.

Although the repeating pattern of HHK was present in H³K4b and H³K8b, N-terminal lysine residues were removed in the highly branched polymer, H³K8b. Reduction in the number of lysine residues in the terminal branches of H³K8b may lead to decreased binding of siRNA and increase the amount of siRNA in the cytoplasm compared to that in the nucleus. By adding a single lysine to each terminal branch of H³K8b (eight lysine residues total per polymer), the efficacy of the new polymer ((+K)H³K8b) in reducing the target mRNA was significantly impaired compared to that of H³K8b. A smaller polymer sequence (i.e., those not having the added lysine to each terminal branch) that accomplishes siRNA transport is advantageous in synthesizing polymers more readily. The idea that binding modulates siRNA release is consistent with the finding that a carrier peptide with increased binding to siRNA is less effective as a carrier for siRNA. (Simeoni F, Morris M C, Heitz F, Divita G. Insight into the mechanism of the peptide-based gene delivery system MPG: implications for delivery of siRNA into mammalian cells. Nucleic Acids Res 2003; 31:2717-2724.). Nevertheless, the vast amount of HK carriers with varying abilities to bind nucleic acids were ineffective carriers of siRNA.

Non-limiting examples of HK polymers according to the present disclosed embodiments include, but are not limited to, one or more polymers selected from the group consisting of HKP, HKP(+H), HKP, HKP(+H), H³K4b, H³K8b, and (-HHHK)H³K8b. Other modifications may be made by those skilled in the art within the scope of this disclosed embodiments. For example, ligands such as, e.g., peptides, aptamers, antibodies and carbohydrates such as hyaluronic acid (HA) targeting the CD44 receptor may be added to the polymer(s) within the scope of the present disclosed embodiments. Additionally, polymers in size between and including a histidine-lysine polymer and (—HHHK)H³K8b polymer are within the scope of the present disclosed embodiments. Further, a fifth or sixth amino acid may be removed from H³K8b and still be within the scope of the present disclosed embodiments.

### Synthesis of Histidine-Lysine Copolymers

Synthesis of histidine-lysine copolymers is well known in the art (see e.g., U.S. Pat. Nos. 7,163,695, and 7, 772,201). Briefly, polypeptides may be prepared by any method known in the art for covalently linking any naturally occurring or synthetic amino acid to any naturally occurring or synthetic amino acid in a polypeptide chain which may have a side chain group able to react with the amino or carboxyl group on the amino acids so as to become covalently attached to the polypeptide chain.

For example, but not by way of limitation, branched polypeptides can be prepared as follows: (1) the amino acid to be branched from the main polypeptide chain can be prepared as an N-α-tert-butyloxycarbonyl (Boc) protected amino acid pentafluorophenyl (Opfp) ester and the residue within the main chain to which this branched amino acid will be attached can be an N-Fmoc-. α,γ-diaminobutyric acid; (2) the coupling of the Boc protected amino acid to diaminobutyric acid can be achieved by adding 5 grams of each precursor to a flask containing 150 ml DMF, along with 2.25 ml pyridine and 50 mg dimethylaminopyridine and allowing the solution to mix for 24 hours; (3) the polypeptide can then be extracted from the 150 ml coupling reaction by mixing the reaction with 400 ml dichloromethane (DCM) and 200 ml 0.12N HCl in a 1 liter separatory funnel, and allowing the phases to separate, saving the bottom aqueous layer and re-extracting the top layer two more times with 200 ml 0.12 N HCl; (4) the solution containing the polypeptide can be dehydrated by adding 2-5 grams magnesium sulfate, filtering out the magnesium sulfate, and evaporating the remaining solution to a volume of about 2-5 ml; (5) the dipolypeptide can then be precipitated by addition of ethyl acetate and then 2 volumes of hexanes and then collected by filtration and washed two times with cold hexanes; and (6) the resulting filtrate can be lyophilized to achieve a light powder form of the desired dipolypeptide. Branched polypeptides prepared by this method will have a substitution of diaminobutyric acid at the amino acid position which is branched. Branched polypeptides containing an amino acid or amino acid analog substitution other than diaminobutyric acid can be prepared analogously to the procedure described above, using the N-Fmoc coupled form of the amino acid or amino acid analog.

Polypeptides of the transport polymer can also be encoded by viral DNA and be expressed on the virus surface. Alternatively, histidine could be covalently linked to proteins through amide bonds with a water soluble dicarbodiimide.

The HK transport polymer may also include a polypeptide—“synthetic monomer” copolymer. In these embodiments, the transport polymer backbone may comprise covalently linked segments of polypeptide and segments of synthetic monomer or synthetic polymer. The synthetic monomer or polymer may be biocompatible and/or biodegradable. Examples of synthetic monomers include ethylenically or acetylenically unsaturated monomers containing at least one reactive site for binding to the polypeptide. Suitable monomers as well as methods for preparing a polypeptide—“synthetic monomer” copolymer are described in U.S. Pat. No. 4,511,478, for “Polymerizable compounds and methods for preparing synthetic polymers that integrally contain polypeptides,” by Nowinski et al, which is herein incorporated by reference. Where the transport polymer comprises a branched polymer, synthetic monomer or polymer may be incorporated into the backbone(s) and/or branch(es). Furthermore, a backbone or branch may include a synthetic monomer or polymer. Finally, in this embodiment, the branching monomers may be branching amino acids or branching synthetic monomers. Branching synthetic monomers may include for example, ethylenically or acetylenically unsaturated monomers containing at least one substituent reactive side-group. Additionally these side groups may consist of peptide (or non-peptide) sequences that are able to bind to select targets on cell membranes—providing the ability to specifically deliver siRNAs or other nucleotides to specific cell types within an organism.

Transport HK polymers in accordance with the present disclosed embodiments may be synthesized by methods known to those skilled in the art. By way of non-limiting example, certain HK polymers discussed herein may be synthesized as follows. The Biopolymer Core Facility at the University of Maryland may be used to synthesize for example, the following HK polymers on a Ranin Voyager solid-phase synthesizer (PTI, Tucson, Ariz., USA): (1) H²K4b (83 mer; molecular weight 11137 Da); (2) H³K4b (71 mer; MW 9596 Da); (3) HK4b (79 mer; MW 10896 Da); (4) H³K8b (163 mer; MW 23218 Da); (5) H³K8b (166 mer; MW 23564 Da); (6) (-HHHK)H³K8b (131 mer; MW 18901 Da); (7) (-HHHK)H³K8b (134 mer; MW 19243 Da); (8) ((K+) H³K8b (174 mer; MW 24594 Da). The structures of certain branched polymers are shown in U.S. Pat. No. 7,772,201. The polymers with four branches (e.g. H³K4b, HK4b) may be synthesized by methods known in the art. The sequence of synthesis for highly branched polymers with eight terminal branches may be as follows: (1) RGD or other ligand (if present); (2) the 3-lysine core; (3) histidine-rich domain; (4) addition of a lysine; and (5) terminal branches. The RGD sequence may be initially synthesized by the instrument followed by three manual couplings with (fmoc)-Lys-(Dde)(the lysine core). The (Dde) protecting groups may be removed during the automatic deprotection cycle. To the lysine core, activated amino acids that comprise the histidine-rich domain may then be added sequentially by the instrument. A (fmoc)-Lys-(fmoc) amino acid was added to the histidine-rich domain and the fmoc protecting groups were then removed. To the a and c amine groups of this lysine, activated amino acids of the terminal branches may then be added. The peptide is cleaved from the resin and precipitated by methods known in the art.

By way of non-limiting example, polymers of the disclosed embodiments may be analyzed as follows. Polymers may be first analyzed by high-performance liquid chromatography (HPLC; Beckman, Fullerton, Calif., USA) and might not be further purified if HPLC reveals that the purity of polymers is 95% or greater. The polymers may be purified on an HPLC column, for example with System Gold operating software, using a Dynamax 21-4.times.250 mm C-18 reversed phase preparative column with a binary solvent system. Detection may be at 214 nm. Further analyses of the polymers may be performed for example, using a Voyager matrix-assisted laser desorption ionization time-of-flight (MALDI-TOF) mass spectrometer (Applied Biosystems, Foster City, Calif., USA) and amino acid analysis (AAA Laboratory Service, Boring, Oreg., USA). Transfection agents such as, SuperFect (Qiagen, Valencia, Calif.), Oligofectamine (Invitrogen, Carlsbad, Calif.), Lipofectamine 2000 (Invitrogen), and Lipofectamine (Invitrogen) may be used according to the manufacturers' instructions. DOTAP liposomes may be prepared by methods known in the art.

Suitable HKP copolymers are described in WO/2001/047496, WO/2003/090719, and WO/2006/060182. HKP copolymers form a nanoparticle containing an siRNA molecule, typically 100-400 nm in diameter. HKP and HKP(+H) both have a lysine backbone (three lysine residues) where the lysine side chain ε-amino groups and the N-terminus are coupled to [KH₃]₄K (for HKP) or KH₃KH₄[KH₃]₂K (for HKP(+H). The branched HKP carriers can be synthesized by methods that are well-known in the art including, for example, solid-phase peptide synthesis.

Formation of Nanoparticles Containing Copolymer, and siRNA

Nanoparticles advantageously are formed for administration to a subject. Various methods of nanoparticle formation are well known in the art. See, e.g., Babu et al., IEEE Trans Nanobioscience, 15: 849-863 (2016).

The addition of an acid such as 1N HCl to the siRNA composition prior to mixing with the histidine/lysine (HKP) copolymer composition modulates properties of the nanoparticles.

Nanoparticles may be formed using a microfluidic mixer system, in which one or more siRNA molecules are mixed with one or more HKP copolymers at a fixed flow rate. The flow rate can be varied to vary the size of the nanoparticles produced. Methods are described in Example 1 below.

### Transfection

Branched carriers comprising histidine and lysine are useful for transfection of plasmids. (See Chen Q R, Zhang L, Stass S A, Mixson A J. Branched co-polymers of histidine and lysine are efficient carriers of plasmids. Nucleic Acids Res 2001; 29:1334-1340.) In these branched co-polymers, the lysine and histidine component forms a complex with and partially neutralizes the negative charge of the plasmid DNA. In addition, the histidine component, with a pKa of about 6.0, buffers and aids in the release of plasmid DNA from endosomal vesicles. In general, linear HK peptides are ineffective for delivery of siRNA. In the present disclosed embodiments cover novel, highly branched HK polymers that are unexpectedly effective carriers of siRNA. The HK polymers of the present disclosed embodiments are advantageous, for example, in that they are less toxic and provide a more efficacious delivery of siRNA than other polymers.

The HK polymers of the present disclosed embodiments may be useful, for example, for in vitro delivery of siRNA to the interior of a cell. These polymers may, however, also have in vivo applications. These methods all include contacting a transfection complex with one or more cells to deliver the siRNA. The transfection complex includes at least one transport polymer and siRNA. The transport polymer includes histidine and lysine.

In general, a cell to be transfected includes, but is not limited to, any animal, plant or bacterial cell that is susceptible to intracellular delivery of siRNA using the transfection complex of the present disclosed embodiments either in vitro or in vivo. For example, suitable cellular targets include, without limitation, epithelial cells, endothelial cells, keratinocytes, fibroblasts, muscle cells, hepatocytes, blood cells such as T lymphocytes, B lymphocytes, monocytes, macrophages, neutrophils, eosinophils, megakaryocytes, granulocytes, various stem or progenitor cells, in particular hematopoietic stem or progenitor cells, e.g., as obtained from bone marrow, umbilical cord blood, peripheral blood, fetal liver, and the like. In certain aspects, the cell is selected from the group consisting of lung cells, liver cells, endothelial cells, muscle cells, skin cells, hematopoietic stem cells and tumor cells.

According to certain embodiments, the cells include one or more cells selected from the group consisting of transformed, recombinant, malignant, and primary cell lines. By way of non-limiting example, cells according to the present disclosed embodiments may include one or more cells selected from SVR-bag4, MDA-MB-435, C6 and HUVEC (human umbilical endothelial vein) cell lines.

With plasmid-based therapy, nuclear import is important for transcription to occur and this appears to be a rate-limiting step in several cell lines. (Pollard H, Remy J S, Loussouarn G, Demolombe S, Behr J P, Escande D.) Polyethylenimine but not cationic lipids promotes transgene delivery to the nucleus in mammalian cells. (J Biol Chem 1998; 273:7507-7511; Zabner J, Fasbender A J, Moninger T, Poellinger K A, Welsh M J. Cellular and molecular barriers to gene transfer by a cationic lipid. J Biol Chem 1995; 270:18997-19007.) Because nuclear import is unnecessary for siRNA to degrade its target mRNA, it is believed that the polymers of the present disclosed embodiments will be effective as carriers of siRNA in most cell lines.

Methods of transfecting cells in accordance with the present disclosed embodiments may also include forming the transfection complex and allowing the transfection complex to stand for about 15 minutes to about 1½ hours, or from about 15 to about 45 minutes at approximately room temperature before contacting the transfection complex with cells.

Transport polymers, that include histidine and lysine in accordance with the present disclosed embodiments include one or more HK carriers that are effective for transporting siRNA, including for example, polymers having between six and 10 terminal branches. According to certain embodiments, the transport polymer of the present disclosed embodiments includes eight terminal branches and a histidine-rich domain. According to certain embodiments, the transport polymer comprises a terminal branch having a sequence of -HHHKHHHKHHHKHHHKHHH- or a version thereof. Non-limiting examples of transport polymers in accordance with the present disclosed embodiments include one or more polymers selected from H³K8b and structural analogs, such as H³K8b including one or more other ligand(s), (—HHHK)H³K8b, and the like.

Transport polymers of the present disclosed embodiments may optionally include one or more stabilizing agents. Suitable stabilizing agents would be apparent to those skilled in the art in view of this disclosure. Non limiting examples of stabilizing agents in accordance with the present disclosed embodiments include polyethyleneglycol (PEG) or hydroxypropylmethylacrylimide (HPMA).

Transport polymers of the present disclosed embodiments may optionally include one or more targeting ligands. Suitable targeting ligands would be apparent to those skilled in the art in view of this disclosure.

The disclosed embodiments are further directed to compositions, which include transfection complexes of the present disclosed embodiments. Such compositions may include for example, one or more intracellular delivery components in association with the HK polymer and/or the siRNA. The intracellular delivery component may include for example, a lipid (such as cationic lipids), a transition metal or other components that would be apparent to those skilled in the art.

In certain embodiments, transfection complex compositions include a transport polymer (which may act as an intracellular delivery component) and siRNA. In these embodiments the transport polymer may act as the intracellular delivery component without need for additional delivery components, or may act in conjunction with other delivery components.

In other embodiments, the transfection complex compositions may include (i) the transport polymer, (ii) at least one intracellular delivery component in association with the transport polymer, and (iii) siRNA in association with the intracellular delivery component and/or the transport polymer. Methods of making these compositions may include combining (i) and (ii) for a time sufficient for the transport polymer and the siRNA to associate into a stable complex. Components (i), (ii) and (iii) may also be provided in a suitable carrier, such as a pharmaceutically acceptable carrier. In embodiments that include an intracellular delivery component other than the transport polymer, the transport polymer may interact with an intracellular delivery component, such as a liposome, through non-covalent or covalent interactions. The transport polymer may interact with siRNA through non-covalent or covalent interactions. Alternatively, the transport polymer need not interact directly with the siRNA, but rather, the transport polymer may react with an intracellular delivery component(s), which in turn interacts with the siRNA, in the context of the overall complex.

The present disclosed embodiments further include assays for determining an effective carrier of siRNA for transfection into cells. These assays include mixing siRNA with a transport polymer to form a transfection complex; contacting the transfection complex with one or more cells; and detecting the presence or absence of siRNA activity within the cells. In certain embodiments, the siRNA is directed toward beta-galactosidase.

### Delivery Components

Intracellular delivery components of the presently disclosed embodiments comprise the transport polymer itself. Where intracellular delivery components other than the transport polymer are utilized such delivery components may be viral or non-viral components. Suitable viral intracellular delivery components include, but are not limited to, retroviruses (e.g., murine leukemia virus, avian, lentivirus), adenoviruses and adeno-associated viruses, herpes simplex viruses, rhinovirus, Sendai virus, and Poxviruses. Suitable non-viral intracellular delivery components include, but are not limited to, lipids and various lipid-based substances, such as liposomes and micelles, as well as various polymers known in the art.

Suitable lipids include, but are not limited to, phosphoglycerides, sphingolipids, phosphatidylcholine, phosphatidylethanolamine, phosphatidylserine, phosphatidylinositol, phosphatidic acid, palmitoyloleoyl phosphatidyleholine, lysophosphatidylcholine, lysophosphatidylethanolamine, dipalmitoylphosphatidylcholine, dioleoylphosphatidylcholine, distearoylphosphatidylcholine, dilinoleoylphosphatidylcholine, glycosphingolipid, amphipathic lipids. The lipids may be in the form of unilamellar or multilamellar liposomes.

The intracellular delivery component may include, but are not limited to, a cationic lipid. Many such cationic lipids are known in the art. A variety of cationic lipids have been made in which a diacylglycerol or cholesterol hydrophobic moiety is linked to a cationic headgroup by metabolically degradable ester bond, for example: 1,2-Bis(oleoyloxy)-3-(4-′-trimethylammonio)propane (DOTAP), 1,2-dioleoyl-3-(4′-trimethylammonio)butanoyl-sn-glycerol (DOTB), 1,2-dioleoyl-3-succinyl-sn-glycerol choline ester (DOSC) and cholesteryl (4′-trimethylammonio)butanoate (ChoTB). Other suitable lipids include, but are not limited to, cationic, non-pH sensitive lipids, such as: 1,2-dioleoyl-3-dimethyl-hydroxyethyl ammonium bromide (DORI), 1,2-dioleyloxypropyl-3-dimethyl-hydroxyethyl ammonium bromide (DORIE), and 1,2-dimyristyloxypropyl-3-dimethyl-hydroxyethyl ammonium bromide (DMRIE). Other non-pH-sensitive, cationic lipids include, but are not limited to: O,O′-didodecyl-N-[p-(2-trimethylammonioethyloxy)benzoyl]-N,N,N-trimethylammonium chloride, Lipospermine, DC-Chol (3 beta [N-(N′,N″-dimethylaminoethane) carbonyl]cholesterol), lipopoly(L-lysine), cationic multilamellar liposomes containing N-(alpha-trimethylamnmonioacetyl)-didodecyl-D-glutamate chloride (TMAG), TransfectACE™ (1:2.5 (w:w) ratio of DDAB which is dimethyl dioctadecylammonium bromide and DOPE) (Invitrogen) and lipofectAMINE™ (3:1 (w:w) ratio of DOSPA which is 2,3-dioleyloxy-N-[20([2,5-bis [(3-amino-propyl)amino]-1-oxypentyl] amino)et-hyl]-N,N-dimethyl-2,3-bis(9-octadecenylo-xy)-1-propanaminium trifluoroacetate and DOPE) (Invitrogen). Other suitable lipids are described in U.S. Pat. No. 5,965,434, for “Amphipathic PH sensitive compounds and delivery systems for delivering biologically active compounds,” by Wolff et al.

Cationic lipids that may be used in accordance with the presently disclosed embodiments comprise, but are not limited to, those that form liposomes in a physiologically compatible environment. Suitable cationic lipids include, but are not limited to cationic lipids selected from the group consisting of 1,2-dioleythyloxypropyl-3-trimethyl ammonium bromide; 1,2-dimyristyloxypropyl-3-dimethyl-hydroxyethyl ammonium bromide; dimethyldioctadecyl ammonium bromide; 1,2-dioleoyl-3-(trimethylammonium)propane (DOTAP); 3. beta.N-(N′,N′-dimethylaminoethane)carbamoyl] cholestero-1 (DC-cholesterol); 1,2 dioleolyl-sn-glycero-3-ethylphosphocholine; 1,2 dimyristoly-sn-glycero-3-ethylphosphocholine; [1-(2,3-diol-eyloxy)propyl]-N,N,N-trimethyl-ammonium chloride (DOTMA); 1,3-dioleoyloxy-2-(6carhoxys-permyl) propylamide (DOSPER); 2,3-dioleyloxy-N-[2(spermine-carboxyamido)et-hyl]-N,N, dimethyl-1-propanamoniumtrifluoroacetate (DOSPA); and 1,2-dimyristyloxypropyl-3-dimethyl-hydroxyethyl ammonium bromide (DMRIE).

Cationic lipids may be used with one or more helper lipids such as diloleoylphosphatidylethanolamine (DOPE) or cholesterol to enhance transfection. The molar percentages of these helper lipids in cationic liposomes are between about 5 and 50%. In addition, pegylated lipids, which can prolong the in vivo half-life of cationic liposomes, can be present in molar percentages of between about 0.05 and 0.5%.

Compositions in accordance with the disclosed embodiments may alternatively include one or more components to enhance transfection, to preserve reagents, or to enhance stability of the delivery complex. For example, in certain embodiments stabilizing compounds such as polyethylene glycol can be covalently attached to either the lipids or to the transport polymer.

Compositions of the disclosed embodiments may also suitably comprise various delivery-enhancing components known in the art. For example, the composition may comprise one or more compounds known to enter the nucleus or ligands subject to receptor-mediated endocytosis, and the like. For example, the ligand may comprise a fusogenic viral peptide to disrupt endosomes, allowing the nucleic acid to avoid lysosomal degradation. Other examples of delivery-enhancing components include, but are not limited to, nuclear proteins, adenoviral particles, transferrin, surfactant-B, anti-thrombomodulin, intercalating agents, hemagglutinin, asialoglycoprotein, chloroquine, colchicine, integrin ligands, LDL receptor ligands, and viral proteins to maintain expression (e.g. integrase, LTR elements, rep proteins, oriP and EBNA-1 proteins) or viral components that interact with the cell surface proteins (e.g. ICAM, HA-1, MLV's gp70-phosphate transporter, and HIV's gp120-CD4). Delivery enhancing components can be covalently or non-covalently associated with the transport polymer, the intracellular delivery component, or the pharmaceutical agent. For instance, delivery to a tumor vasculature can be targeted by covalently attaching a -RGD- or -NGR- motif. This could be accomplished using a peptide synthesizer or by coupling to amino groups or carboxyl groups on the transport polymer with a water-soluble di-carbodiimide (e.g., 1-ethyl-3-(3-dimethyaminopropyl)carboiimide). Both of these methods are known to those familiar with the art.

Compositions of the present disclosed embodiments may suitably include a transition metal ion, such as a zinc ion. The presence of a transition metal in the complexes of the disclosed embodiments may enhance transfection efficiency.

### Administration

The pharmaceutical compositions described herein may be administered to subjects, including human subjects, by any mode of administration that is conventionally used to administer compositions. Thus, the compositions can be in the form of an aerosol, dispersion, solution, or suspension and can be formulated for inhalation, intramuscular, oral, sublingual, buccal, parenteral, nasal, subcutaneous, intradermal, or topical administration. The term parenteral as used herein includes percutaneous, subcutaneous, intravascular (e.g., intravenous), intramuscular, or intrathecal injection or infusion techniques and the like.

As used herein, an effective dose of a composition is the dose required to produce a protective immune response in the subject to whom the pharmaceutical composition is administered. A protective immune response in the present context is one that prevents or ameliorates a variety of diseases or disorders.

The composition may be administered one or more times. An initial measurement of an desired effect to the composition may be made by measuring one or more compounds in the circulation or tissue samples of the recipient subject. Methods of measuring a variety of compounds in this manner are also well known in the art, as is an appropriate dose effective in preventing or inhibiting the occurrence, or treating (alleviate a symptom to some extent, preferably all of the symptoms) of a disease state.

The pharmaceutically effective dose depends on the type of disease, the composition used, the route of administration, the type of mammal being treated, the physical characteristics of the specific mammal under consideration, concurrent medication, and other factors that those skilled in the medical arts will recognize that, generally, an amount between 0.1 mg/kg and 100 mg/kg body weight/day of active ingredients is administered dependent upon potency of the formulated composition, between about 0.1 mg/kg and about 1.0 mg/kg, between about 1.0 mg/kg and about 2.0 mg/kg, from between about 2.0 mg/kg and 3.0 mg/kg, between about 3.0 and 5.0 mg/kg, between about 5 mg/kg and about 8 mg/kg, between about 8 mg/kg and about 15 mg/kg, between about 15 mg/kg and about 25 mg/kg, between about 25 mg/kg and about 35 mg/kg, between about 35 mg/kg and about 45 mg/kg, between about 45 mg/kg and about 55 mg/kg, between about 55 mg/kg and about 65 mg/kg, between about 65 mg/kg and about 75 mg/kg, between about 75 mg/kg and about 85 mg/kg, between about 85 mg/kg and about 95 mg/kg, and between about 95 mg/kg and about 105 mg/kg.

However, their application has until recently been restricted by the instability and inefficient in vivo delivery of nucleic acids such as siRNA molecules. The methods described herein provide methods of making and using pharmaceutical compositions with a HK copolymer nanoparticle delivery system.

The methods described herein may be used in clinical applications of the siRNA include prophylactic and therapeutic compositions effective against various diseases, especially infectious diseases and oncological indications.

### Treatment of Subjects

The present disclosed embodiments provide methods of treating diseases comprising using the complexes or compositions of the present disclosed embodiments. In particular, methods are provided for treating a patient having a disease, by administering to the patient a therapeutically effective amount of a complex or composition of the present disclosed embodiments. Also encompassed are methods for treating a patient having a disease, by administering to the patient cells that have been transfected by the methods disclosed herein. Examples of genetic and/or non-neoplastic diseases potentially treatable with the complex, compositions, and methods include, but are not limited to the following: adenosine deaminase deficiency; purine nucleoside phosphorylase deficiency; chronic granulomatous disease with defective p47phox; sickle cell with HbS, .beta.-thalassemia; Faconi's anemia; familial hypercholesterolemia; phenylketonuria; ornithine transcarbamylase deficiency; apolipoprotein E deficiency; hemophilia A and B; muscular dystrophy; cystic fibrosis; Parkinsons, retinitis pigmentosa, lysosomal storage disease (e.g., mucopolysaccharide type 1, Hunter, Hurler and Gaucher), diabetic retinopathy, human immunodeficiency virus disease virus infection, acquired anemia, cardiac and peripheral vascular disease, and arthritis. In some of these examples of diseases, the therapeutic gene may encode a replacement enzyme or protein of the genetic or acquired disease, an antisense or ribozyme molecule, a decoy molecule, or a suicide gene product.

Ex vivo and in vivo gene therapy with siRNA could also be used to treat a variety of cancers. These siRNA applications include, without limitation: 1) reducing expression of growth factors, reducing proteins that augment the cell cycle (e.g., KRAS Raf-1, PI-3 kinase, MEK or mTOR), growth factor receptors (e.g., EGFR, Her-2), or proteins critical for supporting cells of the tumor (e.g., VEGF, VEGFR1-2 for tumor endothelial cells); 2) targeting or reducing expression of factors that are anti-apoptotic (e.g., BCL-2 or BCLXL); and 3) targeting proteins or enzymes that reduce immune activation toward tumor (PDL1, PD1 or CTLA4 among others).

The present disclosed embodiments also disclose a method of ex vivo gene therapy comprising: (i) removing a cell from a subject; (ii) delivering a nucleic acid (such as siRNA) to the interior of the cell by contacting the cell with a transfection complex or composition comprising such a transfection complex of the present disclosed embodiments; and (iii) administering the cell comprising the nucleic acid (e.g., siRNA) to the subject.

Recombinant cells may be produced using the complexes of the present disclosed embodiments. Resulting recombinant cells can be delivered to a subject by various methods known in the art. In certain embodiments, the recombinant cells are injected, e.g., subcutaneously. In other embodiments, recombinant skin cells may be applied as a skin graft onto a patient. Recombinant blood cells (e.g., hematopoietic stem or progenitor cells) are preferably administered intravenously. The cells can also be encapsulated in a suitable vehicle and then implanted in the subject. The amount of cells administered depends on a variety of factors known in the art, for example, the desired effect, subject state, rate of expression of the chimeric polypeptides, etc., and can readily be determined by one skilled in the art.

All ranges and ratios disclosed here can and necessarily do describe all subranges and subratios therein for all purposes, and all such subranges and subratios also form part and parcel of the disclosed embodiments. Any listed range or ratio can be easily recognized as sufficiently describing and enabling the same range or ratio being broken down into at least equal halves, thirds, quarters, fifths, tenths, etc. As a non-limiting example, each range or ratio discussed herein can be readily broken down into a lower third, middle third and upper third, etc.

The embodiments disclosed of pharmaceutical formulations may be used alone or in combination with other treatments or components of treatments for other dermatological or nondermatological disorders.

The disclosed embodiments will be better understood by reference to the following examples which are intended for purposes of illustration and are not intended to be interpreted in any way to limit the scope of the appended claims.

The word “exemplary” is used herein to mean “serving as an example, instance, or illustration.” Any embodiment described herein as “exemplary” is not necessarily to be construed as preferred or advantageous over other embodiments.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim in this or any application claiming priority to this application require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 ¶6. It will be apparently to those having skill in the art that changes may be made to the details of the above-described embodiments without departing from the underlying principles of the disclosed embodiments.

While specific embodiments and application of the disclosed embodiments have been illustrated and described, it is to be understood that the disclosed embodiments are not limited to the precise configuration and components disclosed herein. Various modifications, changes, and variations, which will be apparent to those skilled in the art may be made in the arrangement, operation, and details of the methods and systems of the embodiments disclosed herein, including those of the appended claims. Finally, various features of the disclosed embodiments herein may be combined to provide additional configurations which fall within the scope of the disclosed embodiments. The following examples are intended to illustrate the kinetic measures and the efficacy of inhibitory compounds tested, including those in the disclosed embodiments.

### Example 1

HKP-siRNA or HKP(+H)-siRNA nanoparticles were prepared using a NanoAssemblr microfluidic instrument (Precision NanoSystems, Inc.). Specifically, HKP and HKP(+H) stocks were prepared in water and diluted at 2.5 mg/mL, providing an acetate content of HKP of 11 percent, and HKP(+H) of 14 percent. Glacial acetic acid was added to the HKP and HKP(+H) solutions to give a final acetate concentration of 15, 20 or 25 percent in each of the HKP and HKP(+H) solutions. The siRNA stock was prepared in water and diluted at 1 mg/mL. The siRNA and copolymer were mixed in a 1:1 volume ratio at a 12 mL/min and 10 mL/min total flow rate, respectively. Particle size was determined by DLS with Zetasizer Ultra (Malvern Panalytical).

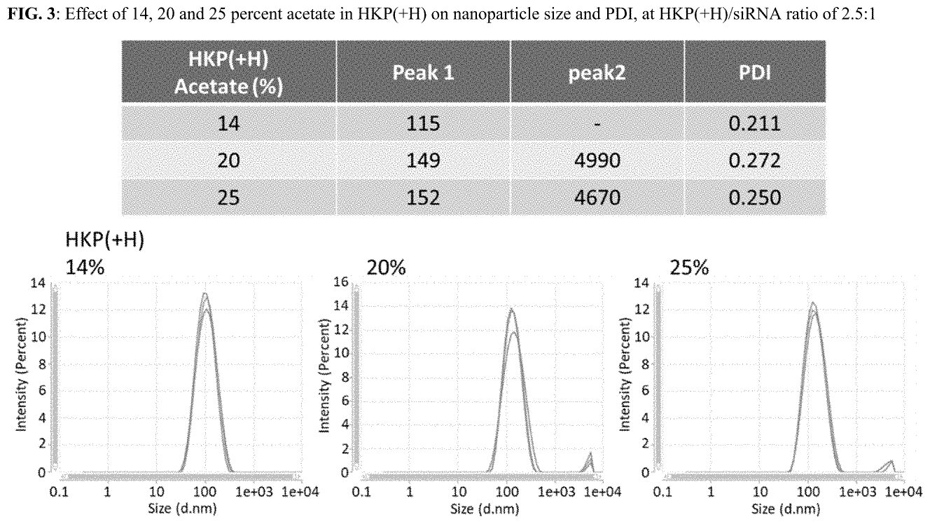

FIG. 2 shows the HKP to siRNA ratio of 2.5 to 1; FIG. 3 shows the same ratio of HKP(+H) to siRNA. Twelve to 18 percent acetate content in the copolymer solution produced reduced nanoparticle size and PDI. Acetate content above 18 percent yielded nanoparticles of a greater range of sizes. The data suggest that an acetate content between about 11 and about 18 percent provides an ideal nanoparticle size and PDI.

### Example 2

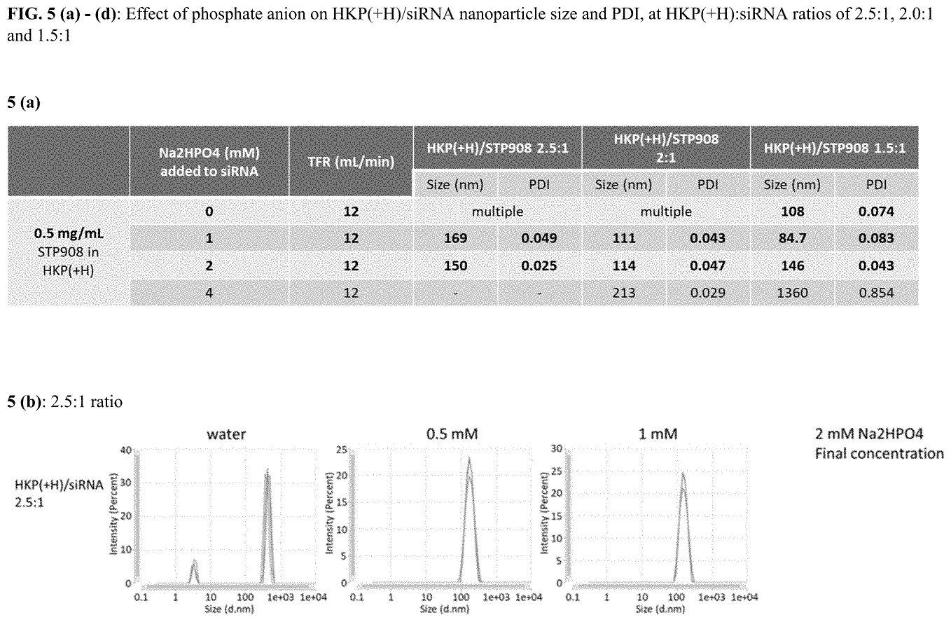

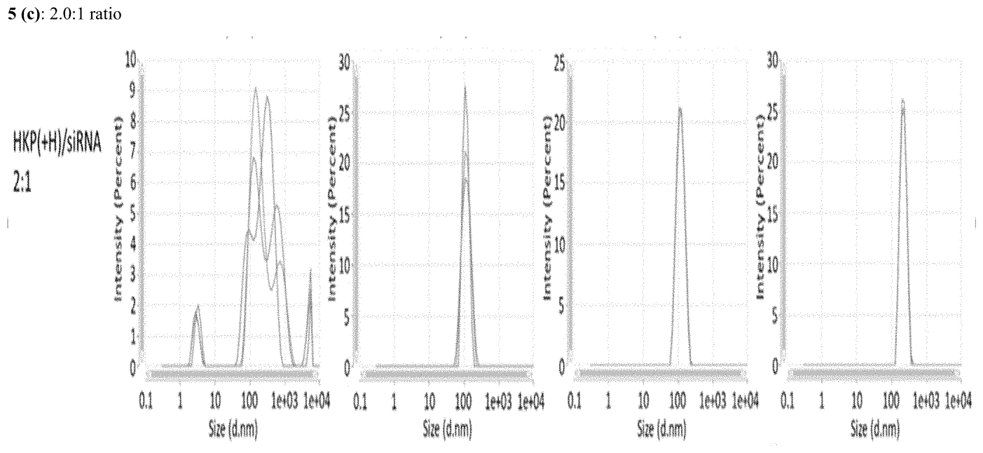

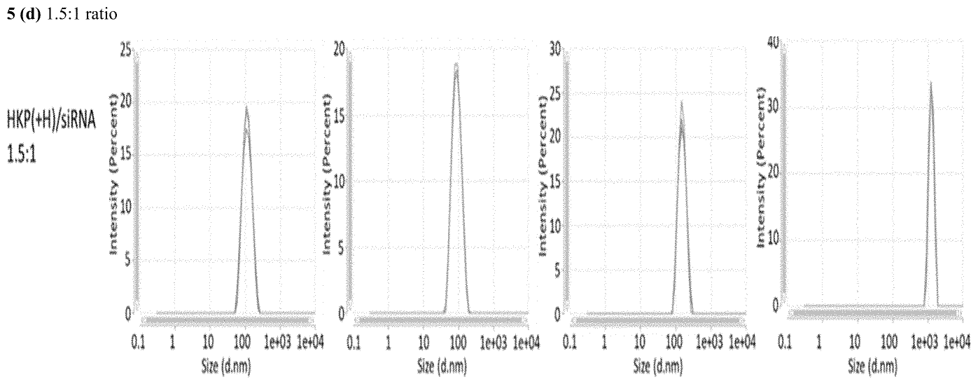

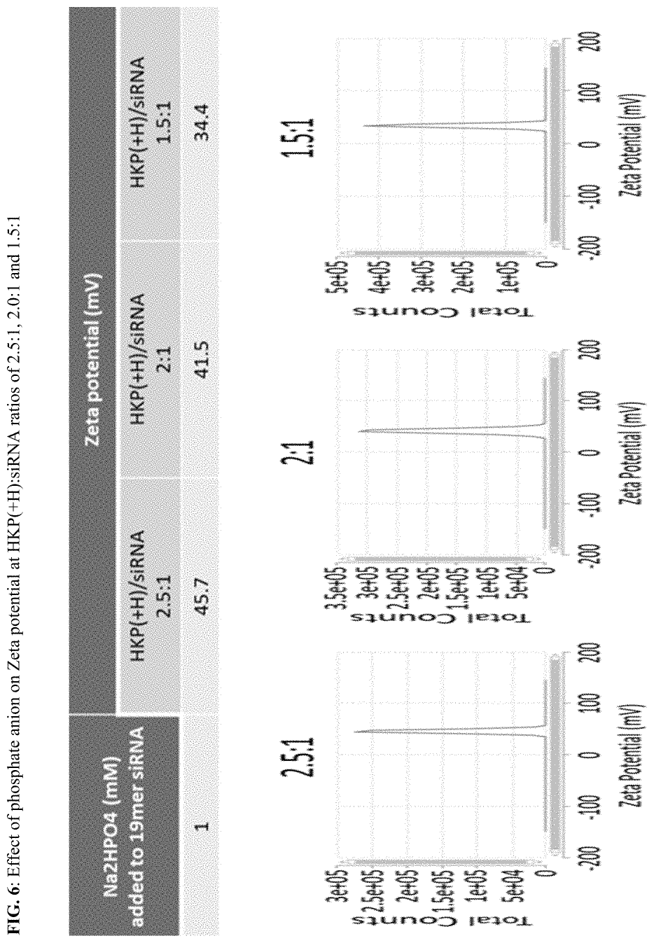

The same protocol as in Example 1 was employed in Example 2 with the addition of a phosphate anion (as 1-2 mM Na₂HPO₄) to the siRNA. This addition enabled the siRNA solution to form, when mixed with the histidine-lysine copolymer, monodisperse nanoparticles for ratios of (HKP(+H) to siRNA) 2:1 and 2.5:1 at a flow rate of 12 mL/min (see FIGS. 4 and 5 (a)-(d)). At the 1.5:1 ratio, the addition of 1 mM Na₂HPO₄ seemed to lower nanoparticle size without affecting PDI. The Zeta potential of the nanoparticles at the 2.5:1, and 2:1 ratios containing 0.5 mM Na₂HPO₄ was roughly 45 and 41 mV, respectively. The effect of storage at 4° C. and −20° C. on nanoparticle size and PDI after addition of phosphate anion is also evident. We saw no effect of the addition of phosphate (Na₂PO₄—dibasic) on Zeta potential FIG. 6.

1. A pharmaceutical composition comprising:
a nanoparticle formulation prepared by microfluidic mixing of (i) a solution comprising a histidine-lysine copolymer and (ii) a solution comprising an effective amount of at least one nucleic acid,
wherein said copolymer solution comprises acetate present in the amount of about 11% to about 20% of the composition, and/or phosphate anion present in the amount of between about 1 and about 2 mM,
wherein at least 40%, at least 45%, at least 50%, at least 55% or at least about 60% of said nanoparticles formed have a diameter in a range selected from the group consisting of between about 40 and about 200 nm, between about 50 and about 150 nm, between about 50 and about 100 nm, and between about 60 and about 90 nm.

2. The composition according to claim 1, wherein the nanoparticles in said composition have a polydispersity index (PDI) selected from the group consisting of between about 0.4 and about 0.3, between about 0.3 and about 0.2, between about 0.2 and about 0.1, between about 0.1 and about 0.05, between about 0.05 and about 0.03, or between about 0.03 and about 0.01.

3. The pharmaceutical composition according to claim 1, wherein the histidine-lysine copolymer is selected from the group consisting of HKP, HKP(+H), HKP, HKP(+H), H³K4b, and H³K8b.

4. The pharmaceutical composition according to claim 1, wherein the nucleic acid is an siRNA molecule.

5. The composition according to claim 4 wherein said siRNA molecule is 18-25 nucleotides long.

6. The pharmaceutical composition according to claim 4, wherein the siRNA reduces the expression of TGFβ1.

7. The pharmaceutical composition according to claim 1, wherein the histidine-lysine copolymer comprises HKP(+H).

8. The pharmaceutical composition according to claim 1, wherein the histidine-lysine copolymer comprises HKP.

9. A method of preparing a pharmaceutical composition comprising
mixing a solution (a) comprising a nucleic acid, and a solution (b) comprising a histidine-lysine copolymer and acetate,
wherein the nucleic acid solution (a) comprises at least one siRNA, and
wherein the histidine-lysine copolymer solution (b) has an acetate content of 11-20% and, optionally, a phosphate anion content of about 1 to about 2 mM.

10. The method according to claim 9 wherein solution (b) has an acetate content selected from the group consisting of: between about 11 and about 20 percent, between about 17 and about 20 percent, between about 14 and about 17 percent, between about 12 and about 14 percent, and between about 11 and about 14 percent.

11. The method according to claim 9, wherein solution (b) comprises HKP.

12. The method according to claim 9, wherein solution (b) comprises HKP(+H).

13. A method of treating a subject having a disease comprising:
administering an effective amount of a pharmaceutical composition according to claim 1, wherein the nucleic acid is an RNA that modulates the production of a protein or peptide of interest, and wherein the disease is ameliorated by the administration of said pharmaceutical composition.

14. The method according to claim 13, wherein the RNA molecule comprises one or more siRNA molecules that inhibit expression of one or more genes associated with the disease.

15. The method according to claim 14 wherein said disease is cancer.

16. The method according to claim 15, wherein said cancer is selected from the group consisting of isSCC, BCC, H&N, liver, NSCLC, other solid tumors, pancreatic, colon, breast, prostate and CNS tumors.

17. The method according to claim 12, wherein said disease is an infection.

18. The method according to claim 13, wherein said subject is a mammal.

19. The method according to claim 18, wherein said mammal is a human.
